(19) United States
(12) Patent Application Publication
(10) Pub. No.: US 2008/0213364 A1
(43) Pub. Date: Sep. 4, 2008

(54) Formulations of Pyridoxal-5'-Phosphate and Methods of Preparation

(75) Inventor: Albert Friesen, Manitoba (CA)

Correspondence Address: MERCHANT & GOULD PC, P.O. BOX 2903, MINNEAPOLIS, MN, 55402-0903, US

(73) Assignee: Medicure International, Inc., St. James, West Indies (BB)

(21) Appl. No.: 11/720,422

(22) PCT Filed: Nov. 28, 2005
(86) PCT No.: PCT/CA05/01810

§ 371 Date: Nov. 26, 2007

Related U.S. Application Data
(60) Provisional application No. 60/630,568, filed on Nov. 26, 2004.

(51) Int. Cl. A61K 9/28; A61K 31/4412; A61P 1/08

(52) U.S. Cl. 424/474; 514/350

(57) ABSTRACT

The present invention provides pharmaceutical compositions for oral administration comprising pyridoxal-5′-phosphate wherein the compositions contain an amount of pyridoxal-5-phosphate of at least 50% w/w and methods of preparing the pharmaceutical compositions. The present invention also provides a pre-blend for the manufacture of a pyridoxal 5′-phosphate oral dosage form comprising pyridoxal 5′-phosphate and microcrystalline cellulose, wherein the pre-blend contains an amount pyridoxal-5-phosphate greater than or equal to 80% w/w.

### FIELD OF INVENTION

The present invention relates to pharmaceutical formulations of pyridoxal-5′-phosphate and methods of preparing the same.

### BACKGROUND

Pyridoxal 5′-phosphate is useful for the treatment and prevention of a variety of diseases such as hypertension, cerebrovascular disorders, cardiovascular disorders and diabetes. See for example U.S. Pat. Nos. 6,051,587; 6,417,204; 6,548,519; 6,586,414; 6,605,612; 6,667,315; 6,780,997; 6,677,356; 6,489,348; and 6,043,259. Pyridoxal 5′-phosphate is commercially available in variety of doses. However, currently available supplements generally deliver lower doses of pyridoxal 5′-phosphate which are too low for the treatment of hypertension, cerebrovascular disorders, cardiovascular disorders and diabetes. As such, it is often necessary for the supplement to be administered several times daily in ordered to achieve suitable therapeutic levels.

The present invention provides novel oral pharmaceutical compositions capable of delivering increased amounts of pyridoxal 5′-phosphate as compared to prior art formulations. The present invention also provides novel pharmaceutical compositions which overcome gastrointestinal side effects associated with the intake of high doses of pyridoxal 5′-phosphate.

### SUMMARY OF INVENTION

In a first aspect, the invention provides a pharmaceutical composition for oral administration comprising: pyridoxal-5′-phosphate or a pharmaceutically acceptable salt thereof, a disintegrant, a binding agent, a lubricant, a glidant and an anti-adherent wherein the composition contains an amount of pyridoxal-5′-phosphate of at least 50% w/w.

In an embodiment of the invention, the pharmaceutical composition further comprises a glidant. The glidant may be colloidal silicon dioxide.

In an embodiment of the invention, the pharmaceutical composition further comprises an anti-adherent. The anti-adherent may be talc.

In an embodiment of the invention, the pharmaceutical composition comprises about 66.3% w/w pyridoxal 5′-phosphate or a pharmaceutically acceptable salt thereof; about 3.0% w/w croscarmellose sodium as the disintegrant; about 4.7% w/w povidone and about 22.2% w/w microcrystalline cellulose as the binding agent; about 0.6% colloidal silicon dioxide as the glidant; about 1.1% w/w magnesium stearate as the lubricant and about 2.3% talc as the anti-adherent.

In an embodiment of the invention, the pharmaceutical composition is in the form of a tablet comprising: (a) a core, wherein said core comprises the pyridoxal-5′-phosphate or pharmaceutically acceptable salt, the disintegrant, the binding agent, and the lubricant; (b) a sealing coat surrounding the core; and (c) an enteric coat surrounding the sealing coat.

In an embodiment of the invention, the pharmaceutical composition is in the form of a tablet comprising: (a) a core, wherein said core comprises the pyridoxal-5′-phosphate or pharmaceutically acceptable salt, the disintegrant, the binding agent, the lubricant, the glidant and the anti-adherent; (b) a sealing coat surrounding the core; and (c) an enteric coat surrounding the sealing coat.

In an embodiment of the invention, the sealing coat is Opadryl-IR-7000 White.

In an embodiment of the invention, the amount of Opadryl-IR-7000 White is between 1 and 10% w/w.

In an embodiment of the invention, the enteric coat is Sureteric YAE-6-18107 White.

In an embodiment of the invention, the amount of Sureteric YAE-6-18107 White is between 1 and 20% w/w.

In a second aspect, the invention provides a pre-blend for the manufacture of a pyridoxal-5′-phosphate oral dosage form comprising: pyridoxal-5′-phosphate or a pharmaceutically acceptable salt thereof and microcrystalline cellulose, wherein the pre-blend contains an amount of pyridoxal-5′-phosphate is greater than or equal to 80% by weight.

In an embodiment of the invention, the pre-blend contains an amount of microcrystalline cellulose is greater than or equal to 10% by weight.

In an embodiment of the invention, the pre-blend comprises about 84.8% w/w of pyridoxal-5′-phosphate of a pharmaceutically acceptable salt and about 15.2% w/w microcrystalline cellulose.

In a third aspect, the present invention provides a method of preparing the pharmaceutical composition comprising pyridoxal-5′-phosphate or a pharmaceutically acceptable salt thereof, a disintegrant, a binding agent, and a lubricant wherein the composition contains an amount of pyridoxal-5′-phosphate of at least 50% w/w, the method comprising the steps of: (1) granulating the pyridoxal 5′-phosphate or the pharmaceutical salt thereof, with the disintegrant, the binding agent, and the lubricant to provide a tableting preparation; and (2) compressing the tableting preparation into a core.

In an embodiment of the invention, step(1) of the method further comprises blending the disintegrant, the binding agent, and the lubricant with a glidant and an anti-adherent.

In an embodiment of the method of the invention, the disintegrant is croscarmellose sodium; the binding agent is a povidone and microcrystalline cellulose, the lubricant is magnesium stearate, the glidant is colloidal silicon dioxide and the anti-adherent is talc.

In a further embodiment of the method of the invention, step (1) comprises the steps of: (a) dissolving 1 to 10% w/w of the povidone in purified water to provide a granulating solution; (b) mixing 50 to 80% w/w of the pyridoxal-5′-phosphate or pharmaceutically acceptable salt with 2 to 15% w/w of a first amount of microcrystalline cellulose to provide a pre-blend; (c) mixing the pre-blend with the granulating solution to provide a first preparation; (d) substantially drying the first preparation; (e) mixing 2 to 15% of a second amount of the microcrystalline cellulose, 3.0% w/w of the croscarmellose sodium, 1 to 5% w/w of the talc and 0.1 to 3% w/w of a glidant to provide a second preparation; and (f) mixing the first and second preparation with 1 to 2% w/w of the magnesium stearate to provide the tableting preparation.

In a still further embodiment of method of the invention, the amount of povidone is about 4.7% w/w; the amount of pyridoxal 5′-phosphate is about 66.30% w/w; the first amount of the microcrystalline cellulose is about 11.9% w/w; the amount of croscarmellose sodium is about 3.0% w/w; the second amount of the microcrystalline cellulose is about 10.3% w/w; the amount of the croscarmellose sodium is about 3.0%; the amount of magnesium stearate is about 1.1% w/w, the amount of talc is about 2.3% w/w; and the amount of colloidal silicon dioxide is about 0.6% w/w.

In an embodiment of the method of the invention, the method further comprises the steps of: (3) applying a sealing coat to the core to provide a sealed core; and (4) applying an enteric coat to the sealed core.

In an embodiment of the method of the invention, the sealing coat is Opadryl-IR-7000 White.

In an embodiment of the method of the invention, the Opadryl-IR-7000 White is applied as a 15% w/w dispersion.

In an embodiment of the method of the invention, the enteric coat is Sureteric YAE-6-18107 White.

In an embodiment of the method of the invention, the Sureteric YAE-6-18107 White is applied as a 15% w/w dispersion.

In a fourth aspect, the present invention provides a method of reducing the incidence of nausea and vomiting associated with the oral administration of pyridoxal 5′-phosphate or a pharmaceutically acceptable salt thereof, said method comprising the step of administering an effective amount of the pharmaceutical composition according to the invention, said pharmaceutical composition comprising pyridoxal-5′-phosphate or a pharmaceutically acceptable salt thereof, a disintegrant, a binding agent, and a lubricant, wherein the composition contains an amount of pyridoxal-5′-phosphate of at least 50% w/w.

### BRIEF DESCRIPTION OF THE FIGURES

FIG. 1 is a flow chart illustrating the granulation steps in the preparation of pyridoxal 5′-phosphate containing cores for the production of enteric coated tablets.

FIG. 2 is a flow chart illustrating the steps in coating the cores for the production of enteric coated tablets.

FIG. 3 is a line graph illustrating the mean pyridoxal 5′-phosphate plasma concentration following a single oral does of a 15, 30, and 60 mg/kg of an enteric coated formulation of pyridoxal 5′-phosphate.

FIG. 4 is a line graph illustrating individual pyridoxal 5′-phosphate plasma concentration upon multiple dosing at 60 mg/kg daily of an enteric coated formulation of pyridoxal 5′-phosphate over a period of seven days.

FIG. 5 is a line graph illustrating individual pyridoxal 5′-phosphate plasma concentration upon multiple dosing at 30 mg/kg daily of an enteric coated formulation of pyridoxal 5′-phosphate over a period of seven days.

FIG. 6 is a line graph illustrating the mean pyridoxal 5′-phosphate plasma concentration following the seventh dose of a 30 mg/kg an enteric coated formulation of pyridoxal 5′-phosphate.

FIG. 7 is a line graph comparing the mean pyridoxal 5′-phosphate plasma concentration following the seventh dose at 30 mg/kg daily of an enteric coated formulation of pyridoxal 5′-phosphate and the individual single-dose plasma profiles at 15, 30 and 60 mg/kg.

### DETAILED DESCRIPTION

Definitions

The term “percentage weight per weight (% w/w)” refers to the weight percentage of the particular compound or excipient relative to the total weight of the composition of which the compound or excipient is a constituent of.

The term “percentage weight per volume (% w/v)” refers to the weight percentage of the particular compound or excipient relative to the total volume of the solution of which the compound or excipient is a constituent of.

The term “particulate” refers to a state of matter that is characterized by the presence of discrete particles, pellets, beads, or granules irrespective of their size, shape, or morphology.

The term “multiparticulate” as used herein means a plurality of discrete, or aggregated, particles, pellets, beads, granules, or mixtures thereof irrespective of their shape, size, or morphology.

The terms “binding agent” or “binder” as used herein means any substance that helps hold a tablet together. A binding agent” or “binder” includes any substance used to cause adhesion of powder particles in tablet granulations.

The term “lubricant” as used herein means any substance used in tablet formulations to reduce friction during tablet compression. The term “lubricant” also includes any substance which permits the compressed tablet to be properly ejected from a tableting machine.

The term “glidant” as used herein means any substance used in tablet formulations to reduce friction during tablet compression or any substance which are used to facilitate the flow of the powders in the tableting process.

The term “anti-adherent” as used herein means any substance which prevents the sticking of tablet formulation ingredients to punches and dies in a tableting machine during production.

The term “exicipient” as used herein means any inert substance combined with an active drug in order to produce a drug dosage form.

The term “colorant” as used herein means any substance used to impart color to pharmaceutical preparations (e.g., tablets).

The terms “sub-coat”, “seal coat” or “sealing coat” as used herein refers to any protective coating and include coatings which are moisture or solvent resistant.

The terms “enteric coat” or “enteric coating” as used herein, means any coating or shell placed on a tablet that breaks up and releases the drug or active ingredient into the intestine rather than the stomach.

Use of the pharmaceutical composition according to the invention facilitates patient compliance. It is well known that there is an inverse relationship between patient compliance and the frequency of the intake of the medication. The higher the frequency of intake of a prescribed medication, the lower the rate of compliance. It is also known that patient compliance is decreased where the prescribed medication is difficult to administer and consumption of the medication is associated with physical discomfort. The pharmaceutical compositions according to the invention promote patient compliance as the compositions provide high doses of pyridoxal 5′-phosphate in a single or twice daily oral dosage form which is sized for easily swallowing.

A limiting factor in the tolerance to high doses of pyridoxal 5′-phosphate is gastrointestinal discomfort characterized mainly by nausea and vomiting. The present invention provides novel pharmaceutical compositions suitable for the oral administration of high doses of pyridoxal 5′-phosphate with minimal gastrointestinal side effects. Furthermore, controlled release assists in maintaining a therapeutic concentration of drug in the body for an extended period of time by controlling its rate of delivery.

The term “disintegrant” as used herein means any substance used in solid dosage forms to promote the disruption of the solid mass into smaller particles which are more readily dispersed or dissolved.

The present invention provides a pharmaceutical composition capable of delivering high doses of pyridoxal 5′-phosphate comprising pyridoxal-5′-phosphate or a pharmaceutically acceptable salt thereof, a disintegrant, a binding agent, a lubricant, a glidant, and an anti-adherent, wherein the composition contains an amount of pyridoxal-5′-phosphate of at least 50% w/w. In a preferred embodiment, the pharmaceutical composition comprises an amount of pyridoxal 5′-phosphate of between 50 and 80% w/w and more preferably about 60% w/w.

Prior art formulations currently available, generally deliver up to 50 mg of pyridoxal 5′-phosphate per dosage form. Accordingly, the prior art formulations must be administered two, three or more times per day to achieve the desired therapeutic levels of pyridoxal 5′-phosphate. In contrast, the pharmaceutical composition of the present invention has a high proportion of pyridoxal 5′-phosphate or a pharmaceutically acceptable salt thereof.

An individual dosage form of the pharmaceutical composition may contain between 250 and 1000 mg of pyridoxal 5′-phosphate. The pharmaceutical composition according to the invention is suitable for once or twice daily administration.

The high proportion of pyridoxal 5′-phosphate or its salt allows the pharmaceutical composition to be provided in a dosage form which is smaller in size than the dosage forms of prior art formulations. Thus, the pharmaceutical composition according to the invention is easy to administer and are especially useful for patients who find it difficult to swallow large tablets or capsules.

The pharmaceutical composition according to the invention may be prepared using either pyridoxal 5′-phosphate or a pharmaceutically acceptable salt thereof. Both the monohydrate and the anhydrous forms of pyridoxal 5′-phosphate are suitable for preparation of the pharmaceutical compositions of the invention. The pyridoxal 5-phosphate may be provided as salt forms with pharmaceutically compatible counterions such as but not limited, to citrate, tartrate, bisulfate, etc. The pharmaceutically compatible salts may be formed with many acids, including but, not limited to, hydrochloric, sulfuric, acetic, lactic, tartaric, malic, succinic, etc. The salt forms tend to be more soluble in aqueous or other protonic solvents than the corresponding free base forms.

Preferably the disintegrant is croscarmellose sodium. The croscarmellose sodium may constitute about to 2 to 10% w/w of the pharmaceutical composition. Preferably the binding agent is microcrystalline cellulose and a povidone. The microcrystalline cellulose is preferably a microcrystalline cellulose having a particle size of about 0.100 mm such as, but not limited to, Avicel PH 102. The microcrystalline cellulose may constitute about 4 to 30% w/w of the pharmaceutical composition. The povidone is preferably a povidone having a K value of 27-30 such as PVP K30. The lubricant may be magnesium stearate. The anti-adherent may be talc. The glidant may be colloidal silicon dioxide.

In a preferred embodiment, the pharmaceutical composition comprises between 1 to 10% w/w of povidone, 2 to 10% w/w croscarmellose sodium, 1 to 2% w/w magnesium, 1 to 5% w/w talc and 0.1 to 3% w/w colloidal silicon dioxide.

In a further preferred embodiment of the invention, the pharmaceutical composition comprises about 66.3% w/w pyridoxal 5′-phosphate or a pharmaceutically acceptable salt thereof; about 22.2% w/w microcrystalline cellulose; about 3.0% croscarmellose sodium, about 4.7% w/w povidone, about 1.1% w/w magnesium stearate; 2.3% w/w talc; and about 0.6% w/w colloidal silicon dioxide.

The pharmaceutical composition according to the invention may further comprise additional pharmaceutically acceptable carriers, dispersants and excipients. Suitable excipients include fillers such as sugars, including lactose, sucrose, mannitol, or sorbitol, or cellulose preparations such as, maize starch, wheat starch, rice starch, potato starch, gelatin, gum tragacanth, methyl cellulose, hydroxypropylmethyl-cellulose, sodium carboxymethylcellulose, and/or polyvinylpyrrolidone. Disintegrating agents may include cross-linked polyvinylpyrrolidone, agar, or alginic acid or a salt thereof such as sodium alginate. The pharmaceutical composition also may comprise suitable solid or gel phase carriers or excipients. Examples of such carriers or excipients include, but are not limited to, calcium carbonate, calcium phosphate, various sugars, starches, cellulose derivatives, gelatin, and polymers such as polyethylene glycols. Further excipients may comprise anti-adhesives such as talc, colloidal silicon dioxide, titanium dioxide, calcite, microcrystalline cellulose, metallic stearates, and barium sulphates. The composition can also include a granulation binder such as, but limited to, alginic acid.

A sealing coat or sub-coat protects the tablet ingredients from the water in the aqueous enteric coating dispersion to assure the stability of the dosage form. The sub-coat comprises a resin such as shellac, zein, and the like and is applied to the dosage form by well known methods. Sub-coats used in sugar coating processes usually consist of alcoholic solutions (approximately 10-30% solids) of resins such as shellac, zein, cellulose acetate phthalate, or polyvinyl acetate phthalate. Shellac is preferably used in the form of a shellac-based formulation containing polyvinylpyrrolidone. Other suitable polymeric solutions can be used as a sub-coat, such as Opadry® IR-7000 White or a copolymer of dimethylaminoethyl methacrylate and methacrylic acid ester (Eudragit®).

Materials useful for preparing enteric coatings for pharmaceuticals are well-known. These most commonly are pH-sensitive materials which are relatively insoluble and impermeable at the pH of the stomach, but which are more soluble and permeable at the pH of the small intestine and colon. Any coating material which modifies the release of the active ingredient in the desired manner may be used. In particular, coating materials suitable for use in the practice of the invention include but are not limited to polymer coating materials, such as cellulose acetate phthalate, cellulose acetate trimaletate, hydroxypropyl methylcellulose phthalate, polyvinyl acetate phthalate, ammonio methacrylate copolymers such as Eudragit® RS and RL, poly acrylic acid and poly acrylate and methacrylate copolymers such as Eudragit® S and L, polyvinyl acetaldiethylamino acetate, hydroxypropyl methylcellulose acetate succinate, shellac; hydrogels and gel-forming materials, such as carboxyvinyl polymers, sodium alginate, sodium carmellose, calcium carmellose, sodium carboxymethyl starch, polyvinyl alcohol, hydroxyethyl cellulose, methyl cellulose, gelatin, starch, and cellulose based cross-linked polymers in which the degree of crosslinking is low so as to facilitate adsorption of water and expansion of the polymer matrix, hydroxypropyl cellulose, hydroxypropyl methylcellulose, polyvinylpyrrolidone, crosslinked starch, microcrystalline cellulose, chitin, arninoacryl-methacrylate copolymer (Eudragit® RS-PM), pullulan, collagen, casein, agar, gum arabic, sodium carboxymethyl cellulose, polyvinylpyrrolidone, anionic and cationic hydrogels, polyvinyl alcohol having a low acetate residual, a swellable mixture of agar and carboxymethyl cellulose, copolymers of maleic anhydride and styrene, ethylene, propylene or isobutylene, pectin, polysaccharides such as agar, acacia, karaya, tragacanth, algins and guar, polyacrylamides, polyethylene oxides, diesters of polyglucan, crosslinked polyvinyl alcohol and poly N-vinyl-2-pyrrolidone, sodium starch glucolate; hydrophilic polymers such as polysaccharides, methyl cellulose, sodium or calcium carboxymethyl cellulose, hydroxypropyl methyl cellulose, hydroxypropyl cellulose, hydroxyethyl cellulose, nitro cellulose, carboxymethyl cellulose, cellulose ethers, polyethylene oxides, methyl ethyl cellulose, ethylhydroxy ethylcellulose, cellulose acetate, cellulose butyrate, cellulose propionate, gelatin, collagen, starch, maltodextin, pullulan, polyvinyl pyrrolidone, polyvinyl alcohol, polyvinyl acetate, glycerol fatty acid esters, polyacrylamide, polyacrylic acid, copolymers of methacrylic acid or methacrylic acid (e.g. Eudragit®), other acrylic acid derivatives, sorbitan esters, natural gums, lecithins, pectin, alginates, ammonia alginate, sodium, calcium, potassium alginates, propylene glycol alginate, agar, and gums such as arabic, karaya, locust bean, tragacanth, carrageens, guar, xanthan, scleroglucan and mixtures and blends thereof. The thickness of the coating is adjusted to give the desired delay property. In general, thicker coatings are more resistant to erosion and, consequently, yield a longer delay.

In an embodiment of the invention, the pharmaceutical composition is in the form of a tablet comprising: (a) a core, wherein said core comprises the pyridoxal-5′-phosphate or pharmaceutically acceptable salt thereof, the disintegrant, the binding agent, the lubricant, the glidant and the anti-adherent; (b) a sealing coat surrounding the core; and (c) an enteric coat surrounding the sealing coat.

The sealing coat and the enteric coat ensure that the core containing the pyridoxal 5′-phosphate is able to pass through the stomach intact and be selectively absorbed in the intestine. The enteric coat is pH dependent and is preferentially soluble in the relatively alkaline conditions of the intestine as opposed to the acidic conditions of the stomach. The sealing coat prepares the tablet core surface for the application of the enteric coating to ensure maximum efficiency of the enteric coating with minimal disintegration of the core in the stomach. Sealing and enteric coats are well known in the art. Any suitable combination of sealing and enteric coats can be used to prepare the pharmaceutical compositions according to the invention so long as dissolution of the pyridoxal 5′-phosphate core is preferentially limited to the intestine. In a preferred embodiment of the invention, the sealing coat is Opadryl IR-7000 White and constitutes between 1 and 10% w/w and preferably, about 3.1% w/w of the total composition. The enteric coat is preferably Sureteric YAE-6-18107 White and constitutes about 1 to 20% w/w and preferably, 10.2% w/w of the total composition.

The absorption of the coated embodiments of the pharmaceutical compositions is preferentially limited to the intestine. The pharmaceutical compositions selectively and efficiently dissolve in the relatively alkaline environment of the intestine. Preferably, the pharmaceutical compositions have a dissolution profile of greater than 80%, and more preferably a dissolution profile of greater than 90%, at 45 minutes according to the United States Pharmacopoeia dissolution test in a 0.05M phosphate buffered solution having a pH of 6.8. Absorption of the pharmaceutical compositions in the stomach is minimal. The structural integrity of the coated embodiments of the pharmaceutical compositions of the invention is minimally affected by the acidic conditions of the stomach. Preferably, the pharmaceutical compositions have a dissolution profile of less than 10% and more preferably, a dissolution profile of less than 1% at 120 minutes according to the United States Pharmacopoeia dissolution test in 0.1N HCl.

The pharmaceutical compositions according to the present invention provide improved pyridoxal 5′-phosphate bioavailability. Preferably, in vivo oral intake of between 15 and 60 mg/kg of the composition produces an average plasma level of between 0.1 and 2 mg/l of pyridoxal 5′ phosphate in the period from 2 hours after intake to 24 hours after intake. In vivo oral intake of between 15 and 60 mg/kg of the composition preferably produces a maximum plasma level (Cmax) of pyridoxal 5′-phosphate of between 2 and 6 mg/l.

In a second aspect, the present invention provides a pre-blend useful in the manufacture of a pyridoxal 5′-phosphate oral dosage form. Powdered preparations of pyridoxal-5′-phosphate suffer poor flowability. As a consequence, it is difficult to prepare tablets of pyridoxal 5′-phosphate in a consistent manner. Because powdered pyridoxal 5′-phosphate does not tend to disperse evenly, it is difficult to uniformly blend and granulate pyridoxal 5′-phosphate with other ingredients (i.e. excipients) prior to tableting.

Where it is desirable to produce a tablet having a high concentration of pyridoxal 5′-phosphate, it is necessary to granulate the pyridoxal 5′-phosphate in order to alter its physical properties into a material that can flow. Good flow properties are essential for tableting since the powder has to be able to flow into the die cavity in which the tablet will be formed with punches. If the powder does not flow evenly and quickly, it is difficult to control tablet weights. Poor flow properties also necessitates the use of very slow compression speeds which are impractical for commercial purposes.

The present invention provides a pre-blend comprising pyridoxal-5′-phosphate or a pharmaceutically acceptable salt thereof, and microcrystalline cellulose, wherein the pre-blend contains an amount of pyridoxal 5′-phosphate is greater than or equal to 80% by weight. The flow characteristics of the pre-blend, as compared to powdered pyridoxal 5′-phosphate alone, allows for improved ease in handling and in blending the active ingredient with other ingredients such as but not limited to disintegrants, binding agents, lubricants.

In a preferred embodiment, the pre-blend comprises an amount of microcrystalline cellulose of at least 10% by weight. In a further preferred embodiment, the pre-blend comprises about 84.8% w/w of pyridoxal 5′-phosphate or a pharmaceutically acceptable salt thereof and 15.2% w/w of microcrystalline cellulose. In yet a further preferred embodiment, the microcrystalline cellulose is a microcrystalline cellulose having a particle size of about 0.100 mm such as but not limited to, Avicel PH 102.

The pre-blend according to the invention is especially usefully for in the manufacture of oral dosage forms of pyridoxal 5′-phosphate such as tablets and capsules.

In a third aspect, the invention provides a method of preparing the pharmaceutical composition according to the invention which comprises pyridoxal-5′-phosphate or a pharmaceutically acceptable salt thereof, a disintegrant, a binding agent, a lubricant, a glidant and an anti-adherent wherein the composition contains an amount of pyridoxal-5′-phosphate of at least 50% w/w. The method comprises the steps of: (1) granulating the pyridoxal 5′-phosphate or the pharmaceutical salt thereof, with the disintegrant, the binding agent, the lubricant, the glidant, and the anti-adherent to provide a tableting preparation; and (2) compressing the tableting preparation into a core.

In a further embodiment, step (1) of the method comprises the steps of: (a) dissolving 1 to 10% w/w of the povidone in purified water to provide a granulating solution; (b) mixing 50 to 80% w/w of the pyridoxal-5′-phosphate or pharmaceutically acceptable salt with 2 to 15% w/w of a first amount of microcrystalline cellulose to provide a pre-blend; (c) mixing the pre-blend with the granulating solution to provide a first preparation; (d) substantially drying the first preparation; (e) mixing 2 to 15% of a second amount of the microcrystalline cellulose, 3.0% w/w of the croscarmellose sodium, 1 to 5% w/w of the talc and 0.1 to 3% w/w of a glidant, to provide a second preparation; and (f) mixing the first and second preparation with 1 to 2% w/w of the magnesium stearate to provide the tableting preparation.

In a preferred embodiment of the invention, step (a) comprises the step of dissolving about 47% w/w of the povidone in purified water to provide the granulating solution. The povidone is preferably a povidone having a K value of between 27 and 30 and is more preferably PVP K30.

In the preferred embodiment of the invention, step (b) comprises the preparation of a pyridoxal 5′-phosphate containing pre-blend by mixing about 66.3% w/w of the pyridoxal-5′-phosphate (or a pharmaceutically acceptable salt thereof) powder with about 11.9% w/w of a first amount of the microcrystalline cellulose. The microcrystalline cellulose may preferably be a microcrystalline cellulose having a particle size of about 0.100 mm, and more preferably the microcrystalline cellulose is Avicel PH102. The pre-blend can be mixed using a ribbon blender.

In the preferred embodiment of the invention, step (c) comprises mixing the pre-blend with the granulating solution to provide a first preparation using a ribbon blender.

In the preferred embodiment, step (d) comprises the wet first preparation is then substantially dried using a forced air drying oven set at 45° C. In some circumstances, it may be desirable to size the first preparation after drying, by passing it through a 12 mesh screen.

In the preferred embodiment, step (e) comprises the provision of a second preparation by mixing about 10.3% w/w of a second amount of the microcrystalline cellulose, about 3.0% w/w of the croscarmellose sodium, about 2.3% w/w of the talc as the anti-adherent, and about 0.6% w/w of the colloidal silicon dioxide as the glidant. In some circumstances, it may be desirable to size the second granulation preparation by passing it through a 16 mesh screen.

In the preferred embodiment, step (f) comprises mixing together the first and second preparation with about 1.1% w/w of the magnesium stearate, to provide the tableting preparation using a diffusive blender.

Step (2) of the method, compression of the tableting preparation into tablets, can be accomplished using tableting methods and apparatus known in the art. Preferably, the tablets are prepared using a rotary tablet press and plain, round, standard, concave, 11 mm tablet tool.

In another embodiment of the invention, the method for preparing the pharmaceutical compositions according to the invention further comprises following steps (1) and (2), the steps of: (3) applying a sealing coat to the core to provide a sealed core; and (4) applying an enteric coat to the sealed core.

Preferably, the sealing coat is applied as a 15% w/w dispersion of Opadryl-IR-7000 White and the enteric coat is applied as 15% w/w dispersion of Sureteric YAE-6-18107 White. A side vented perforated coating pan or other suitable device can be used to apply the coatings by conventional methods.

In a fourth aspect, the present invention further provides a method of reducing the incidence of nausea and vomiting associated with the oral administration of pyridoxal 5′-phosphate or a pharmaceutically acceptable salt thereof, said method comprising the step of administering an effective amount of pyridoxal 5′-phosphate in a controlled release, delayed release, or a combination of a controlled release and delayed released oral pharmaceutical composition.

By controlled release is meant any formulation technique wherein release of the active substance from the dosage from is modified to occur at a slower rater than that from an immediate release product, such as a conventional swallow tablet or capsule.

By delayed release is meant any formulation technique wherein release of the active substance from the dosage form is modified to occur at a later time than that from a conventional immediate release product. The subsequent release of active substance from a delayed release formulation may also be controlled as defined above.

Such controlled release formulations are preferably formulated in a manner such that release of the pyridoxal 5′-phosphate is effected predominantly during the passage through the stomach and the small intestine, and delayed release formulations are preferably formulated such that release of active substance is avoided in the stomach and is effected predominantly during passage through the small intestine. The small intestine is suitably the duodenum, the ileum or the jejunum.

In a preferred embodiment, the controlled release or delayed release pharmaceutical composition is a pharmaceutical composition according to the invention, comprising: pyridoxal-5′-phosphate or a pharmaceutically acceptable salt thereof, a disintegrant, a binding agent, a lubricant, a glidant and an anti-adherent, wherein the composition contains an amount of pyridoxal-5′-phosphate of at least 50% w/w, wherein the composition is in the form of a tablet comprising: (a) a core, wherein said core comprises the pyridoxal-5′-phosphate, the disintegrant, the binding agent, the lubricant, the glidant and the anti-adherent; (b) a sealing coat surrounding the core; and (c) an enteric coat surrounding the seating coat.

In a further preferred embodiment, the controlled release or delayed release pharmaceutical composition is a pharmaceutical composition according to the invention, comprising: about 66.3% w/w pyridoxal 5′-phosphate or a pharmaceutically acceptable salt thereof; about 3.0% w/w croscarmellose sodium as the disintegrant; about 4.7% w/w povidone and about 22.2% w/w microcrystalline cellulose as the binding agent, about 1.1% w/w magnesium stearate as the lubricant, about 2.3% talc as the anti-adherent, and about 0.6% colloidal silicon dioxide as the glidant.

Any of the coated pharmaceutical compositions of the invention can be used to reduce the incidence of nausea and vomiting associated with the oral administration of pyridoxal 5′-phosphate or a pharmaceutically acceptable salt thereof.

The pharmaceutical compositions generally are administered in an amount effective for treatment or prophylaxis of a specific indication or indications. It is appreciated that optimum dosage will be determined by standard methods for each treatment modality and indication, taking into account the indication, its severity, route of administration, complicating conditions and the like. A therapeutically effective dose further refers to that amount of the compound sufficient to result in amelioration of symptoms associated with such disorders. Techniques for formulation and administration of the compounds of the instant application may be found in “Remington's Pharmaceutical Sciences,” Mack Publishing Co., Easton, Pa., latest edition. For administration to mammals, and particularly humans, it is expected that the daily dosage level of the active agent will be 100 to 1000 mg, typically around 500 mg. The physician in any event may determine the actual dosage which will be most suitable for an individual and will vary with the age, weight and response of the particular individual. The above dosages are exemplary of the average case. There can, of course, be individual instances where higher or lower dosage ranges are merited, and such are within the scope of this invention.

Although the invention has been described with reference to illustrative embodiments, it is to be understood that the invention is not limited to these precise embodiments, and that various changes and modifications may be effected therein by one skilled in the art. All such changes and modifications are intended to be encompassed in the appended claims.

### EXAMPLE ONE

### Pyridoxal 5′-Phosphate Enteric Coated Tablet Formulation and Method of Preparation

Table 1 sets out the ingredients and relative amounts for the preparation of enteric coated tablets of pyridoxal 5′-phosphate (265 mg per tablet). As set out in table 1, one batch yields 20,000 tablets. The batch size can be scaled up or down by increasing or decreasing the relative amounts proportionately.

TABLE 1
Formulation for Enteric Coated Pyridoxal 5′-phosphate Tablets

| | % | mg/ | |
|---|---|---|---|
| Ingredient | w/w | tablet | g/batch |
| Granulation Phase | | | |
| Pyridoxal 5′-phosphate Powder | 66.3 | 265 | 5300 |
| Microcrystalline Cellulose (Avicel PH102) | 11.9 | 47.5 | 950 |
| Povidone (K-30) | 4.7 | 18.75 | 375 |
| Sub-Total: | 82.8 | 331.25 | 6625 |
| Purified Water (for PVP granulation solution) | | qs | 1500 |
| Additional Purified Water (for granulation) | | qs | 150 |
| Tableting Phase | | | |
| Granulation | 82.8 | 331.25 | 6625 |
| Microcrystalline Cellulose (Avicel PH102) | 10.3 | 41 | 820 |
| Croscarmellose Sodium | 3.0 | 12 | 240 |
| Talc | 2.3 | 9 | 180 |
| Colloidal Silicon Dioxide | 0.6 | 2.25 | 45 |
| Magnesium Stearate | 1.1 | 4.5 | 90 |
| Total: | 100.0 | 400 | 8000 |
| Coating Phase | | | |
| Opadry-IR-7000 White (Sealing coat)-15% | 3.1 | 14.1 | 282 |
| dispersion | | | |
| Sureteric YAE-6-18107 White-(Enteric Coat)- | 10.2 | 47.1 | 942 |
| 15% | | | |
| Coated Tablet Total: | 100.0 | 461.2 | |
| Purified Water (for Sealing coat) | | qs | 1598 |
| Purified Water (for Enteric Coat) | | qs | 5338 |

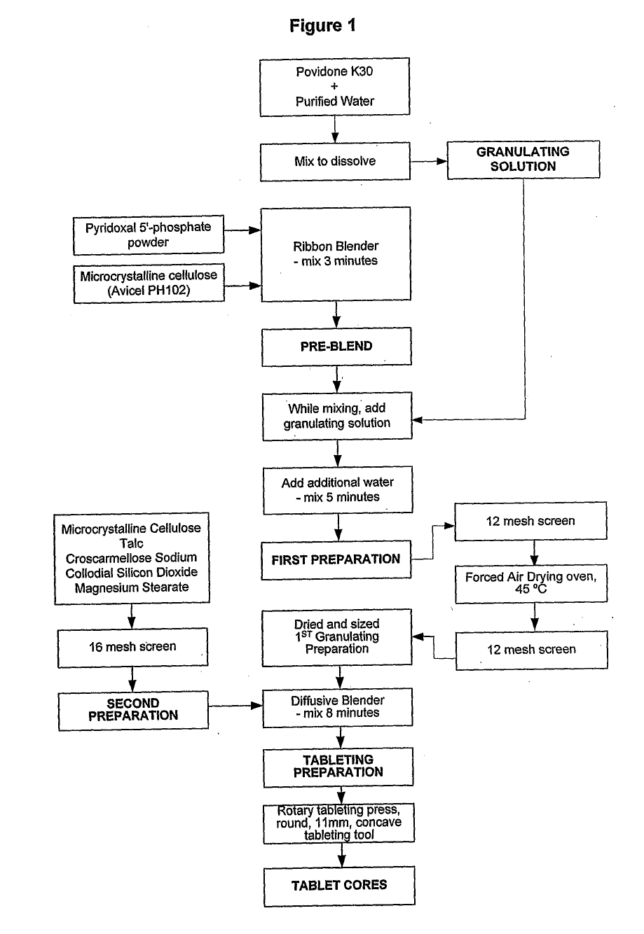

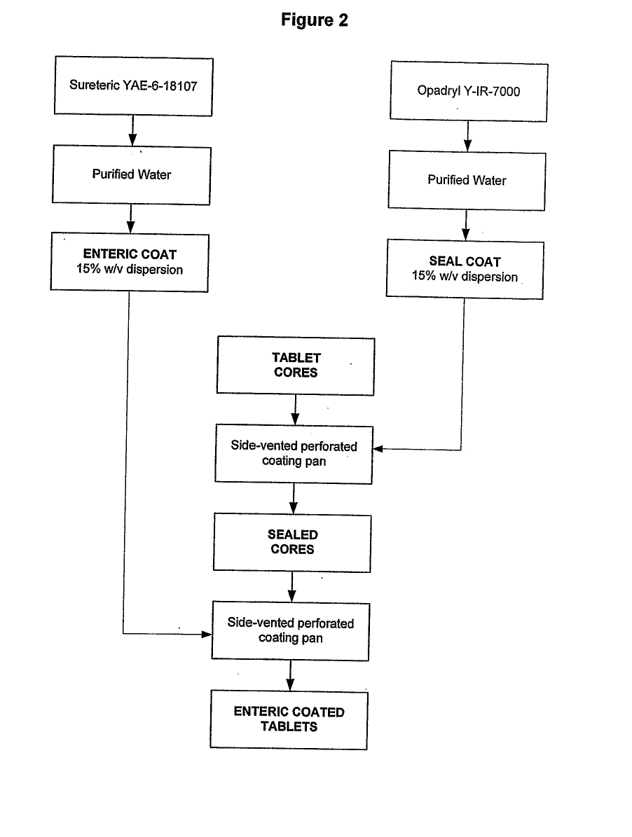

The pyridoxal 5′-phosphate enteric coated tablets are prepared in a three step process: (1) granulation and blending phase, (2) tableting phase, and (3) coating phase. FIGS. 1 and 2 illustrate the steps involved in preparing the tablets.

Granulation and Blending Phase—A granulating solution is prepared by dissolving the Povidone K30 in a suitable amount of purified water. A pyridoxal 5′ phosphate pre-blend is prepared by mixing the pyridoxal 5′-phosphate powder with the first amount of microcrystalline cellulose (Avicel PH 102) for approximately 3 minutes in a ribbon blender. While continuing to mix the pre-blend, the granulating solution is added to form granules. Additional water is added as necessary for the granulating process. The pre-blend and the granulation solution are mixed for approximately 5 minutes. The resulting granules are sized by passing the granules through a 12 mesh screen and then placed on paper lined trays. The granules are then dried in a forced air drying oven at 45° C. The dried granules are sized by passing the granules through a 12 mesh screen. The second amount of microcrystalline cellulose (Avicel PH 102), the croscarmellose sodium, the talc, the colloidal silicon dioxide and magnesium stearate mixed and then passed through a 16 mesh screen. The mixture is then combined with the dried and sized granules and mixed using a diffusive blender for approximately 8 minutes to provide the tableting mixture.

Tableting phase—The tableting preparation is compressed into cores using a rotary tablet press and a plain, 11 mm, round, standard, concave tablet tool.

Coating phase—The sealing coat is prepared by dispersing the Opadryl Y-IR-7000 in a suitable about of purified water to provide a 15% w/w dispersion. A sufficient amount of the Opadryl Y-IR-7000 dispersion is applied the core such that amount of applied Opadryl Y-IR-7000 is about 3.1% w/w relative to the total weight to the finished tablet. The enteric coating is prepared by dispersing the Sureteric YAE-6-18107 in a suitable amount of purified water to provide a 15% w/w dispersion. A sufficient amount of the Sureteric YAE-6-18107 is applied such that the amount of Sureteric YAE-6-18107 is about 10.2% w/w relative to the total weight of the finished tablet. Using a side vented perforated coating pan, the tablet cores are first coated with the sealing coat dispersion. The tablets are then coated with the enteric coat dispersion.

### EXAMPLE TWO

### Stability and Dissolution Analysis of Pyridoxal 5′-Phosphate Enteric Coated Tablet

The dissolution and stability properties of the pyridoxal 5′-phosphate enteric coated tablets were determined using conventional testing methods. The dissolution test was performed in a VanKel Model Vanderkamp 600 (6 spindle) dissolution apparatus equipped with an autosampler, digital thermometer and timer. At the Initial, 0.5, and 1-month testing points, a paddle speed was set up at 75 rpm. After the 3 month testing point, the paddle speed, in view of SUPAC guidelines, was increased to 100 rpm. The sampling volume was 10 ml. A 2-stage dissolution procedure was carried out based on USP 24 <724> method B for enteric coated tablets. The Acid Stage was carried out using 0.1N HCl for 120 minutes at 37° C. followed by the buffer stage at pH 6.8 at 37° C.

At time points Initial, 0.5, and 1-month, storage stability label claim and disintegration data for pyridoxal 5′-phosphate enteric coated tablets were observed within the following specification limits:
Dissolution in 0.1 N HCl: 120 minutes=not more than 10%
Dissolution in pH 6.8 buffer: 45 minutes=no individual tablet is less than 60%;
Dissolution in pH 6.8 buffer: 60 minutes=no individual tablet is less than 60%.

Pyridoxal 5′-phosphate concentration in the buffer stage dissolution was observed to be reduced by approximately 12% in the one-month sample compared to data from time 0.5 month (at 60 minutes).

Table 2 summarizes the stability and dissolution profiles at time points: Initial, 0.5, 1, and 3 months.

| | | Accelerated Studies | | | |
|---|---|---|---|---|---|
| Test/Method | Limit | Initial | 0.5 month | 1 months | 3 months |
| Appearance | (³) | White, bi-convex tablets | White, bi-convex tablets | White, bi-convex tablets | White, bi-convex tablets |
| Tablet weight | 420 mg- | 432.9 mg | 434.7 mg | 435.4 mg | 441.08 mg |
| | 460 mg | | | | |
| Disintegration Time | Tablets | 5 of 6 tablets - no change | 5 of 6 tablets - no change | 4 of 6 tablets - no change | 4 of 6 tablets - no change |
| USP24 <701> in | remain | 1 of 6 swollen on one edge | 1 of 6 swollen on one edge | 2 of 6 swollen on one edge | 2 of 6 swollen on one edge |
| simulated gastric | intact | | | | |
| fluid USP (minus | | | | | |
| pepsin) | | | | | |
| USP24 <701> in | Run and | 14′00″-18′15″ | 11′30″-14′15″ | 13′00″-16′00″ | 6′19″-10′38″ |
| simulated intestinal | report | | | | |
| fluid USP (minus | | | | | |
| pancreatin) | | | | | |
| Visually observe | Run and | Dissolution of film at edges | Dissolution of film at edges | Dissolution of film at edges | Dissolution of film at edges |
| degree of | report | and sides followed by flow | and sides followed by slow | and sides followed by slow | and sides followed by slow |
| dispersion of | degree of | disintegration of the core | disintegration of the core | disintegration of the core | disintegration of the core |
| disintegrated tablet | dispersion | | | | |
| Water content | Run and | 1.8% | 2.7% | 5.3% | 2.5% |
| | report | | | | |
| Dissolution | Run and | Dissolution in 0.1 N HC1 | Dissolution in 0.1 N HC1 | Dissolution in 0.1 N HC1 | Dissolution in 0.1 N HC1 |
| | report⁴ | 120 minutes = 0% label | 120 minutes = 0.1-0.7% | 120 minutes = 0% label | 120 minutes = 0% label |
| | | claim | label claim | claim | claim |
| | | Dissolution in pH 6.8 | Dissolution in pH 6.8 | Dissolution in pH 6.8 | Dissolution in pH 6.8 |
| | | buffer | buffer | buffer | buffer |
| | | 45 minutes = 79.7-116.8% | 45 minutes = 86.8-89.8% | 45 minutes = 67.5-76.5% | 30 minutes = 92.6-94.8% |
| | | label claim | label claim | label claim | label claim |
| | | 60 minutes = 101.2-115.3% | 60 minutes = 87.2-91.0% | 60 minutes = 69.8-83.0% | 45 minutes = 93.7-96.3% |
| | | label claim | label claim | label claim | label claim |
| | | | | | 60 minutes = 95.2-97.3% |
| | | | | | label claim |
| Uniformity of | 85-115% | 97.2% | 95.8% | 97.0% | 97.0% |
| dosage units | (w/w) of | | | | |
| | label | | | | |
| | claim | | | | |
| Assay content of | Release: | 247.3 mg/tablet | 242.1 mg/tablet | 245.3 mg/tablet | 245.3 mg/tablet |
| MC-1 and related | 225-276 mg | | | | |
| substances | | | | | |
| Assay content | Shelf life | not detected | not detected | not detected | not detected |
| related substances | stability in | | | | |
| | progress | | | | |

³Plain white to off-white film-coated bi-convex tablets.
⁴After the two month stability testing point speed of the apparatus has been changed from 75 to 100 RPM (see dissolution summary)

### EXAMPLE THREE

### Bioavailability Analysis for Pyridoxal 5′-Phosphate Enteric Coated Tablet, Single Dose and Multiple Doses

Pharmacokinetics Study—The protocol was designed to first study a single dose of pyridoxal 5′-phosphate at three different dose levels: 15, and 60 mg/kg of body weight. Following evaluation of safety and tolerance, the highest tolerated dose was administered once daily for seven consecutive days. The product was manufactured as enteric-coated tablets each containing 250 mg of pyridoxal 5′-phosphate. The dose was calculated based on the body weight and rounded to the nearest 250 mg.

During the single-dose phase, blood samples were collected prior to dosing and 1, 2, 3, 4, 5, 6, 7, 8, 12, 16 and 24 hours following drug administration. During the multi-dose phase, blood samples were drawn each morning prior to dosing and 2 hours post-dose. Following the last dose on day 7, blood samples were drawn after 1, 2, 3, 4, 5, 6, 7, 8, 12, 16 and 24 hours.

All samples were constantly protected from ultraviolet light, collected in pm-cooled blood collection tubes and kept in an ice bath until centrifugation within twenty minutes of their collection. Plasma samples were separated and divided into two approximately equal aliquots and stored at −70±10° C. pending shipment (over dry ice) to the analytical laboratory.

Electrocardiograms—For the single-dose study, electrocardiograms (ECG's) were recorded prior to dosing and 1, 2 and 12 hours following drug administration. All ECG's were to be recorded within ±15 minutes of the scheduled time.

Vital Signs—Blood pressure, heart rate, respiratory rate and oral body temperature were recorded every day prior to drug administration and 1, 2, 6 and 12 hours post-dose. All vital signs were to be measured within ±15 minutes of their scheduled time.

Pharmacokinetic Analysis—The following pharmacokinetic parameters were calculated: area under the curve from time zero to the last measurable concentration (AUC-T), area under the curve from time zero to infinity (AUC-inf), maximal plasma concentration (Cmax), time of maximal plasma concentration (Tmax), elimination constant (Kel), half-life (T½), mean residence time (MRT), clearance (Cl) and volume of distribution (Vd).

Whenever possible, the area under the plasma concentration-time curve was calculated from time zero to the last measurable concentration (AUC T) using the linear trapezoidal method. AUC-T is expressed as ng·h/ml. AUC-T was also normalized for body weight and expressed as ng·h/ml/kg.

The area under the curve from time zero to infinity (AUC-inf) was calculated as the sum of AUC-T+Clast/Kel where Clast represents the last measurable concentration and Kel the elimination rate constant calculated by linear least squares regression using the data points that best represent the terminal linear phase. AUC-inf is expressed as ng·h/ml. AUC-inf was also normalized for body weight and expressed as ng·h/mg/kg.

Cmax was defined as the highest observed plasma concentration over the 24-hour collection period. Cmax is expressed in ng/ml. Cmax was also normalized for body weight and expressed as ng/ml/kg.

Tmax, was defined as the time where the maximal plasma concentration was reached.

The oral clearance was evaluated by dividing the dose (mg/kg) by AUC-inf (ng·h/mL). Clearance is expressed as L/h/kg. The actual clearance was also calculated without normalization for body weight and expressed as L/h.

The volume of distribution is actually the Vdₐᵣₑₐ calculated by dividing the clearance (L/h/kg) by Kel (h-1). Vd is expressed as L/kg and is not corrected for absolute bioavailability. The actual volume of distribution was also calculated without normalization for body weight and expressed in liters.

The mean residence time (MRT) was defined as the quotient of the area under the first moment curve and AUC-inf. The product of concentration by time is plotted versus time and the area under the first moment curve is calculated. The results are expressed in hours.

Dose Calculation—The dose was calculated based on the body weight and the total dose was rounded to the nearest 250 mg to determine the number of tablets to administer. Descriptive statistics reveal that the actual dose was relatively close to the theoretical dose.

Subjects randomized to the 15 mg/kg group actually received a dose of 14.58±1.11 mg/kg. Those assigned to the 30-mg dose received 30.52±1.44 mg/kg. Subjects in the 60-mg/kg group actually received 59.87±0.91 mg/kg. During the multi-dose phase of the study, the number of tablets was calculated prior to the first drug administration and remained constant throughout the duration of the study, regardless of any change in body weight. Subjects who were to receive 30 mg/kg for seven days actually received 29.93±1.07 mg/kg.

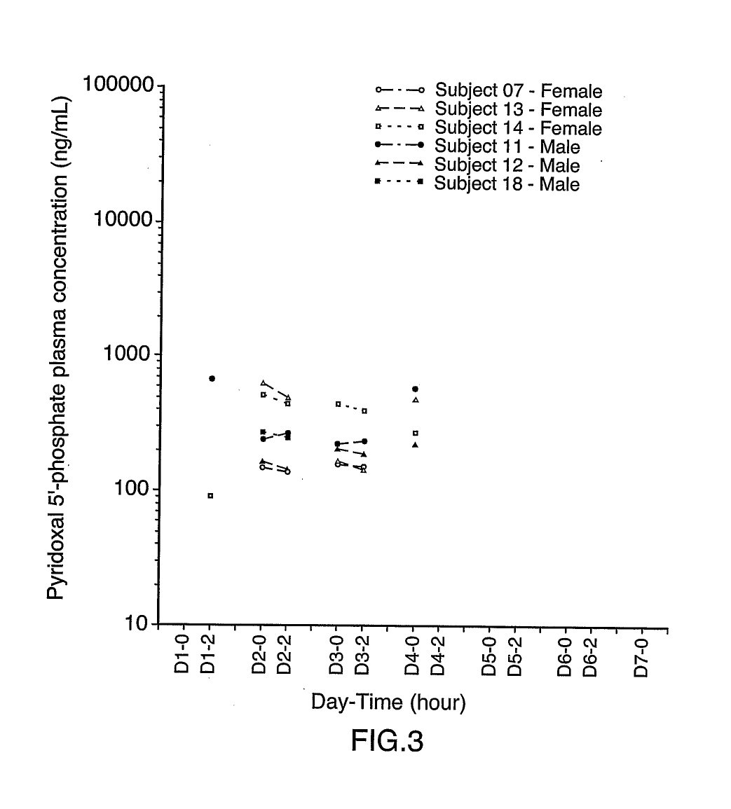

Single Dose Pharmacokinetics—FIG. 3 illustrates the mean plasma concentration following each dose. The pharmacokinetic parameters are summarized in Table 2.

TABLE 2
Summary of Pharmacokinetic Parameters, Single Phase Dose

| | DOSE (mg/kg) | | |
|---|---|---|---|
| | 15 MG/KG | 30 MG/KG | 60 MG/KG |
| NUMBER OF | 4-6 | 5-6 | 6 |
| SUBJECTS | | | |
| AUC-T (ng · h/ml) | 12036 ± 10489 | 25978 ± 45083 | 18685 ± 17831 |
| AUC-INF (ng · h/ml) | 21635 ± 11963 | 34571 ± 50484 | 22503 ± 17036 |
| CMAX (ng/ml) | 2837 ± 2987 | 6426 ± 12423 | 4466 ± 5102 |
| Lag time (h) | 4.5 ± 2.4 | 2.7 ± 1.0 | 2.2 ± 1.2 |
| TMAX (h) | 7.0 ± 2.8 | 4.3 ± 1.2 | 4.5 ± 0.5 |
| T½ (h) | 10.0 ± 4.9 | 20.2 ± 15.6 | 15.3 ± 6.3 |
| MRT (h) | 15.2 ± 5.5 | 11.2 ± 6.1 | 12.1 ± 2.9 |
| CI (L/h) | 66 ± 45 | 128 ± 69 | 250 ± 123 |
| Vd (L) | 2440 ± 2898 | 2876 ± 2269 | 6103 ± 3881 |

Results are expressed as mean ± standard deviation

As illustrated in FIG. 3, following a single dose of pyridoxal phosphate at 15 mg/kg, a mean maximal plasma concentration of 1870±2652 ng/ml was reached after 6 hours. After a single dose of 30 mg/kg, a mean maximal plasma concentration of 5563±12820 ng/ml was reached after 3 hours. Following a single dose of 60 mg/kg, the mean maximal plasma concentration reached 3922±5457 ng/ml after 4 hours.

Examination of the individual curves reveals that the pharmacokinetics of pyridoxal 5′-phosphate is characterized by a large variability. For instance, subject #11 in the 30-mg/kg group reached a maximal plasma concentration of 31,719.5 ng/ml after 3 hours compared to the group average of 5563 ng/ml at 6 hours. Subject #01 and 02 in the 15-mg/kg group had very different maximal plasma concentration. The plasma profile of subject #01 peaked at 6238 ng/ml after 4 hours compared to subject #02 whose whole plasma peak occurred at 132 ng/ml 12 hours post-dose. As shown in Table 2, coefficients of variation varying form 50 to 200% are not unusual.

Multiple Dose—Based on the safety and tolerance profile observed during the single-dose phase of the study, a dose of 60 mg was selected for the multi-dose phase. However, upon multiple dosing, gastrointestinal intolerance developed during the first few days and subjects were dosed for the last time on Day 3. The multi-dose phase was reinitiated using the next highest dose, 30 mg/kg. All subjects completed this part of the study.

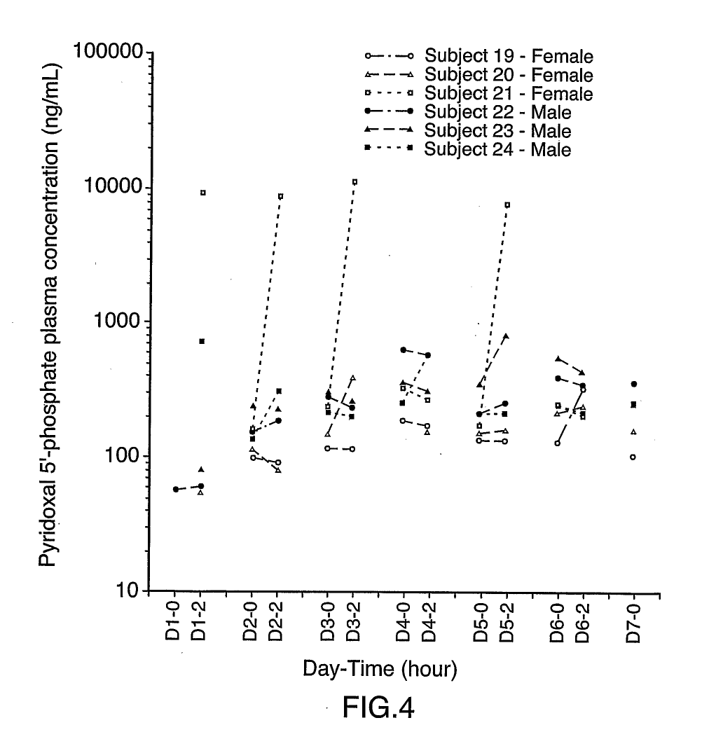

For subjects who participated to the first multi-dose phase at 60 mg/kg, the pre-dose and two-hour post-dose plasma concentrations are summarized in Table 3 and illustrated in FIG. 4.

TABLE 3
Plasma Concentration Upon Multiple Dosing At 60 mg/kg

| Day | Pre-dose (ng/ml) | 2 hours post-dose (ng/ml) |
|---|---|---|
| 1 | 0 ± 0 | 129 ± 273 |
| 2 | 327 ± 197 | 288 ± 149 |
| 3 | 237 ± 117 | 219 ± 102 |
| 4 | 364 ± 157 | — |

Results are expressed as mean ± standard deviation.

Apparent missing data points in FIG. 4 are actually samples below the lower limit of quantitation (50 ng/ml). The lag time previously observed with the single-dose administration could possible explain the lower concentration two hours post-dose compared to the pre-dose.

Table 4 summarizes the descriptive statistics for the second multi-dose phase carried out with an oral dose of 30 mg/kg.

TABLE 4
Plasma Concentration Upon Multiple Dosing at 30 mg/kg

| Day | Pre-dose (ng/ml) | 2 hours post-dose (ng/ml) |
|---|---|---|
| 1 | 9 ± 23 | 1641 ± 3590 |
| 2 | 147 ± 49 | 1558 ± 3390 |
| 3 | 213 ± 73 | 2010 ± 4352 |
| 4 | 342 ± 167 | 336 ± 187 |
| 5 | 202 ± 75 | 1477 ± 2878 |
| 6 | 290 ± 148 | 287 ± 88 |
| 7 | 244 ± 101 | 642 ± 924 |

Results are expressed as mean ± standard deviation

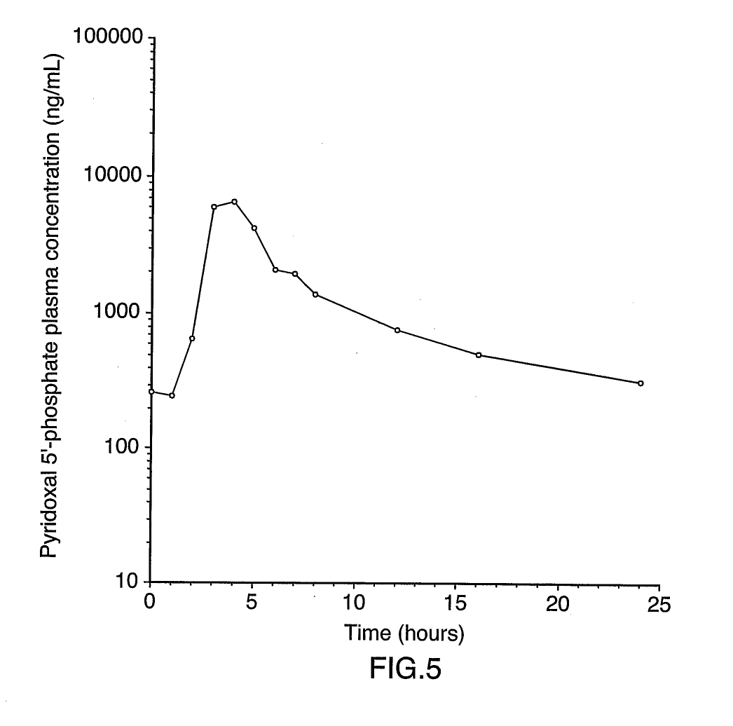

Individual daily plasma concentrations are presented in FIG. 5 and illustrate the large intersubject variability. Subject #21 represents a case where the absorption is particularly important although her pre-dose plasma concentration is similar to that of the other subjects.

These results were submitted to an analysis of variance where the terms of the model included subjects and day of treatment. The pre-dose plasma concentration is significantly different between days (p=0.0011). No significant difference was observed between days with regard to the plasma concentration two hours post-dose (p=0.5284). The analysis of variance including subjects, days and times as the factors revealed a significant difference between pre-dose and two hours post-dose (p=0.0426).

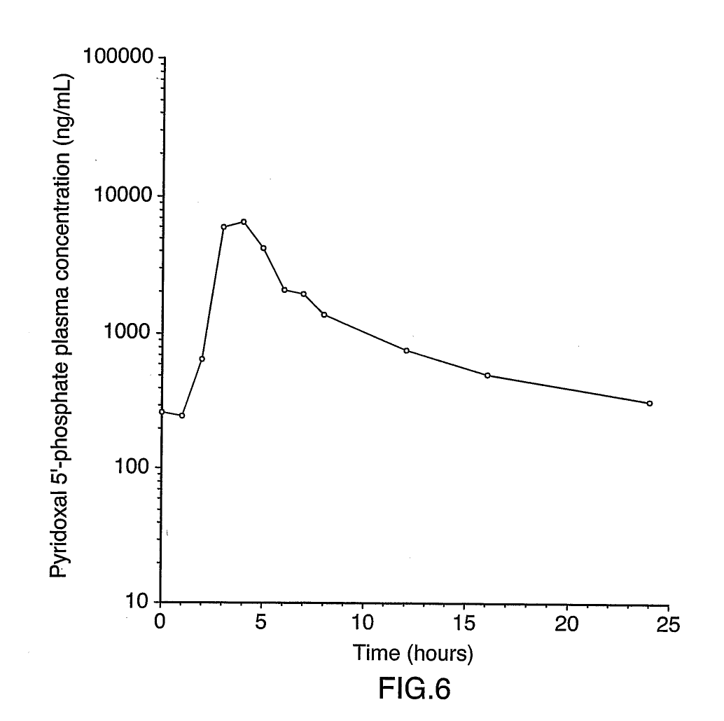

Last Dose of the Multiple Dose (Day 7)—Table 5 summarizes the pharmacokinetic parameters and FIG. 6 illustrates the mean plasma profile. The pharmacokinetic parameters of Table 5 are also characterized by a large variability with coefficients of variation on over 10 The pharmacokinetic parameters calculated on Day 1 were compared to those calculated on Day 7 using an analysis of variance. None of the comparisons revealed a significant difference which would indicate that pyridoxal 5′-phosphate does not accumulate upon multiple dosing, at least at a dose of 30 mg/kg administered as enteric coated tablets.

TABLE 5
Summary of Calculated Pharmacokinetic Parameters
on Day 7 of the Multi-dose Phase at 30 mg/kg

| | DOSE (mg/kg) | |
|---|---|---|
| | 30 MG/KG | |
| AUC-T (ng · h/mL) | 32173 ± 25942 | (5) |
| AUC-INF (ng · h/mL) | 38341 ± 24942 | (5) |
| CMAX (ng/mL) | 7966 ± 6831 | (5) |
| TMAX (h) | 3.6 ± 0.9 | (5) |
| T½ (h) | 23.0 ± 28.0 | (5) |
| MRT (h) | 11.8 ± 5.3 | (5) |
| CI (L/h) | 86 ± 53 | (4) |
| Vd (L) | 4148 ± 5560 | (4) |

Results are expressed as mean ± standard deviation (n)

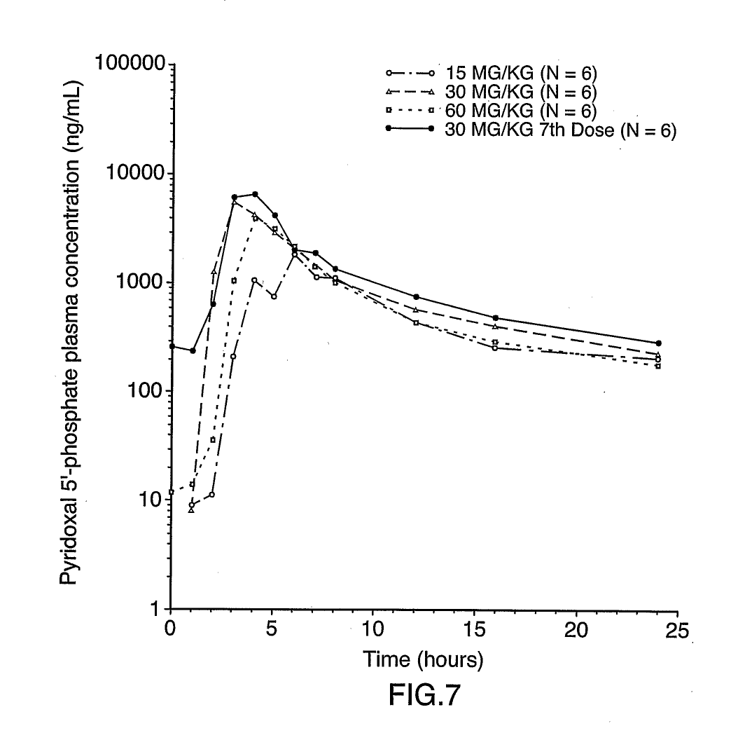

Additional evidence that pyridoxal 5′-phosphate does not accumulate upon multiple dosing is provided in FIG. 7 where the plasma concentration on the seventh day of dosing at 30 mg/kg is plotted on the same graph as the three mean plasma profiles following a single dose of 15 and 60 mg/kg.

Dose Linearity—The relationship between the calculated pharmacokinetic parameters and dose was evaluated by submitting the data to a linear regression analysis where each of the parameters was regressed on the dose (expressed either as mg or mg/kg). The pharmacokinetic parameters were uncorrected as well as normalized for body weight. Table 6 summarizes the regression coefficient (R²), the intercept, the slope and the 95% confidence interval for the slope, for each individual parameter. In these analyzes, the correlation between the uncorrected parameters and the dose expressed in milligrams was evaluated.

TABLE 6
Summary of Regression Analysis, Parameter = Intercept +
Slope × Dose (mg)

| | | | | 95% CONFIDENCE | |
|---|---|---|---|---|---|
| | | | | INTERVAL OF THE | |
| | | | | SLOPE | |
| | | | | LOWER | UPPER |
| PARAMETER | R² | INTERCEPT | SLOPE | LIMIT | LIMIT |
| AUC-t | 0.0213 | 12119 | 2.8298 | −7.3291 | 12.9887 |
| (ng · h/ml) | | | | | |
| AUC-inf | 0.0030 | 23286 | 1.0935 | −10.3228 | 12.5097 |
| (ng · h/ml) | | | | | |
| Cmax (ng/ml) | 0.0183 | 2854 | 0.7249 | −2.0902 | 3.5400 |
| C1 (L/h) | 0.4693 | 19.78 | 0.0541 | 0.0211 | 0.0871 |
| Vd (ml) | 0.1809 | 1485 | 0.9921 | −0.2182 | 2.2025 |
| Tmax (h) | 0.1853 | 6.77 | 0.0006 | −0.0013 | 0.0001 |
| T½ (h) | 0.0000 | 15.90 | 0.00003 | −0.0044 | 0.0043 |
| MRT (h) | 0.0860 | 15.21 | 0.0010 | −0.0028 | 0.0008 |

The regression analysis was also carried out using the normalized parameters (expressed per kg of body weight) and the dose expressed in mg/kg. The results are summarized in Table 7.

TABLE 7
Summary of Regression Analysis, Parameter = Intercept +
Slope × Dose (mg)

| | | | | 95% CONFIDENCE | |
|---|---|---|---|---|---|
| | | | | INTERVAL OF THE | |
| | | | | SLOPE | |
| | | | | LOWER | UPPER |
| PARAMETER | R² | INTERCEPT | SLOPE | LIMIT | LIMIT |
| AUC-t | 0.0083 | 207 | 1.5961 | −7.6550 | 10.8472 |
| (ng · h/ml/kg) | | | | | |
| AUC-inf | 0.0007 | 385 | −0.4816 | −10.6671 | 9.7039 |
| (ng · h/ml/kg) | | | | | |
| Cmax | 0.0064 | 49.6 | 0.3912 | −2.1937 | 2.9761 |
| (ng/ml/kg) | | | | | |
| C1 (L/h/kg) | 0.4640 | 0.2369 | 0.0571 | 0.0219 | 0.0922 |
| Vd (ml/kg) | 0.2051 | 16.67 | 1.2245 | −0.1572 | 2.6062 |
| Tmax (h) | 0.2115 | 7.0 | −0.0495 | −0.1002 | 0.0012 |
| T½ (h) | 0.0054 | 14.23 | 0.0420 | −0.2841 | 0.3681 |
| MRT (h) | 0.0512 | 14.83 | −0.0557 | −0.1932 | 0.0818 |

Gender Differences—The pharmacokinetic parameters calculated during the single-dose phase of the study were compared with regard to gender differences using analyses of variance where the model included dose, gender and the interaction dose by gender. In these analyses, the pharmacokinetic parameters were expressed both its absolute values and normalized per kilogram of body weight. Table 8 summarizes the probability values for the various statistical comparisons.

TABLE 8
ANOVA Probability Values for Gender Differences Following a Single
Dose of 15, 30, or 60 mg/kg.

| Param- | Dose | | Gender | | Dose × Gender | |
|---|---|---|---|---|---|---|
| eter | Absolute | Per kg | Absolute | Per kg | Absolute | Per kg |
| AUCₜ | 0.7170 | 0.7339 | 0.5255 | 0.6745 | 0.3381 | 0.2859 |
| AUC_{inf} | 0.5282 | 0.5536 | 0.4534 | 0.6511 | 0.1109 | 0.0795 |
| Cₘₐₓ | 0.7445 | 0.7802 | 0.5476 | 0.6830 | 0.3371 | 0.2960 |
| C1 | 0.0096 | 0.0205 | 0.5769 | 0.9422 | 0.1494 | 0.3294 |
| Vdarea | 0.1796 | 0.2640 | 0.5511 | 0.3865 | 0.4480 | 0.7508 |

| | | | |
|---|---|---|---|
| Tmax | 0.0462 | 0.3839 | 0.5560 |
| T½ | 0.4238 | 0.6067 | 0.9574 |
| MRT | 0.3202 | 0.2606 | 0.3900 |

No significant differences were observed between men and women with regard to any of the pharmacokinetic parameters. The only significant difference was observed between doses for clearance expressed in absolutes units or normalized by body weight. This highly significant difference relates to the weak linear relationship previously observed. The maximal plasma concentration appears to be reached significantly later following the 15 mg/kg dose.

Discussion—Six subjects (three men and three women) were enrolled in each dose level. All of the subjects completed the single-dose phase of the study. Based on the clinical evaluation, the 60-mg/kg dose appeared to be the highest tolerated dose. This dose was therefore selected for the multi-dose administration. Gastrointestinal intolerance developed and the decision to terminate the study was taken during the third day. Subjects were kept under observation and were discharged on the next day. The multi-dose phase was rescheduled for a similar study using a daily dose of 30 mg/kg.

Enteric-coating the tablets resolved the nausea and vomiting problem observed in the previous study. Subjects could tolerate doses much higher than those used previously.

There is an absence of accumulation of pyridoxal 5′-phosphate upon multiple dosing. The pre-dose plasma concentration appears to stabilize within one or two days and is maintained approximately between 200 and 300 ng/ml.

1. A pharmaceutical composition comprising: at least 50% w/w pyridoxal-5′-phosphate or a pharmaceutically acceptable salt thereof.

2. The pharmaceutical composition according to claim 1, wherein the amount of the pyridoxal-5′-phosphate is at least 50% to about 80% w/w.

3. The pharmaceutical composition according to claim 1, wherein the amount of the pyridoxal-5′-phosphate is about 60% w/w.

4-24. (canceled)

25. A pharmaceutical composition comprising about 66.3% w/w pyridoxal 5′-phosphate or a pharmaceutically acceptable salt thereof; about 3.0% w/w croscarmellose sodium; about 4.7% w/w povidone; about 22.2% w/w microcrystalline cellulose; about 0.6% colloidal silicon dioxide; about 1.1% w/w magnesium stearate; and about 2.3% talc.

26. The pharmaceutical composition of claim 25, wherein the composition is in the form of a tablet comprising: (a) a core, wherein said core comprises the pyridoxal-5′-phosphate or pharmaceutically acceptable salt, the croscarmellose sodium, povidone, microcrystalline cellulose, and magnesium stearate; (b) a sealing coat surrounding the core; and (c) an enteric coat surrounding the seating coat.

27-33. (canceled)

34. The pharmaceutical composition according to claim 26, wherein the composition has a dissolution profile of greater than 80% at 45 minutes according to the United States Pharmacopoeia dissolution test in a 0.05M phosphate buffered solution having a pH of 6.8.

35. The pharmaceutical composition according to claim 26, wherein the composition has a dissolution profile of greater than 90% at 45 minutes according to the United States Pharmacopoeia dissolution test in a 0.05M phosphate buffered solution having a pH of 6.8.

36. The pharmaceutical composition according to claim 26, wherein the composition has a dissolution profile of less than 10% at 120 minutes according to the United States Pharmacopoeia dissolution test in 0.1N HCl.

37. The pharmaceutical composition according to claim 26, wherein the composition has a dissolution profile of less than 1% at 120 minutes according to the United States Pharmacopoeia dissolution test in 0.1N HCl.

38. The pharmaceutical composition according to claim 1, wherein in vivo oral intake of between 15 and 60 mg/kg of the composition produces a maximum plasma level (Cmax) of pyridoxal-5′-phosphate of about 1 to about 8 mg/L.

39. The pharmaceutical composition according to claim 1, wherein in vivo oral intake of between 15 and 60 mg/kg of the composition produces an average plasma level of about 0.1 to about 2 mg/L of pyridoxal-5′-phosphate in the period from 2 hours after intake to 24 hours after intake.

40. A pre-blend composition comprising: at least 80% pyridoxal-5′-phosphate by weight or a pharmaceutically acceptable salt thereof and microcrystalline cellulose.

41-46. (canceled)

47. A method of preparing the pharmaceutical composition according to claim 1, comprising the steps of:
(1) granulating the pyridoxal-5′-phosphate or the pharmaceutical salt thereof, with a disintegrant, a binding agent, and a lubricant to provide a tableting preparation; and
(2) compressing the tableting preparation into a core.

48. The method according to claim 47, wherein step (1) further comprises blending the disintegrant, the binding agent, and the lubricant with a glidant and an anti-adherent.

49-53. (canceled)

54. A method of according to claim 48, wherein step (1) comprises the steps of:
(a) dissolving 1 to 10% w/w of the povidone in purified water to provide a granulating solution;
(b) mixing 50 to 80% w/w of the pyridoxal-5′-phosphate or pharmaceutically acceptable salt with 2 to 15% w/w of a first amount of microcrystalline cellulose to provide a pre-blend;
(c) mixing the pre-blend with the granulating solution to provide a first preparation;
(d) substantially drying the first preparation;
(e) mixing 2 to 15% of a second amount of the microcrystalline cellulose, 3.0% w/w of the croscarmellose sodium, 1 to 5% w/w of the talc and 0.1 to 3% w/w of colloidal silicon dioxide to provide a second preparation; and
(f) mixing the first and second preparation with 1 to 2% w/w of magnesium stearate to provide the tableting preparation.

55-66. (canceled)

67. A method of reducing nausea or vomiting associated with the oral administration of pyridoxal-5′-phosphate or a pharmaceutically acceptable salt thereof, said method comprising the step of administering an effective amount of the pharmaceutical composition according to any one of claims 26.

68. (canceled)

69. The pharmaceutical composition of claim 1, wherein the composition is in the form of a tablet comprising:
(a) a core, comprising the pyridoxal-5′-phosphate or pharmaceutically acceptable salt, a disintegrant, a binding agent, and a lubricant;
(b) a sealing coat surrounding the core; and
(c) an enteric coat surrounding the sealing coat.

70. The pharmaceutical composition according to claim 26, wherein in vivo oral intake of between 15 and 60 mg/kg of the composition produces a maximum plasma level (Cmax) of pyridoxal-5′-phosphate of about 1 to about 8 mg/L.

71. The pharmaceutical composition according to claim 26, wherein in vivo oral intake of between 15 and 60 mg/kg of the composition produces an average plasma level of about 0.1 to about 2 mg/L of pyridoxal-5′-phosphate in the period from 2 hours after intake to 24 hours after intake.
